(19) United States
(12) Patent Application Publication
(10) Pub. No.: US 2023/0301881 A1
(43) Pub. Date: Sep. 28, 2023

(54) PERSONAL CARE COMPOSITIONS WITH ANTIMICROBIAL, HYDRODYNAMIC, AND ANTIOXIDANT NANOPARTICLES

(71) Applicant: EVOQ NANO, INC., Salt Lake City, UT (US)

(72) Inventor: William H. NIEDERMEYER, West Jordan, UT (US)

(21) Appl. No.: 18/126,911

(22) Filed: Mar. 27, 2023

Related U.S. Application Data
(60) Provisional application No. 63/324,464, filed on Mar. 28, 2022.

(51) Int. Cl. A61K 8/19; A61K 8/02; A61K 33/242; A61Q 11/00; A61K 33/38; A61Q 19/00

(52) U.S. Cl. CPC A61K 8/19; A61K 8/025; A61K 33/242; A61Q 11/00; A61K 33/38; A61Q 19/00; A61K 8/0283; A61K 2800/524; A61K 2800/522; A61K 2800/413

(57) ABSTRACT

Personal care compositions contain a carrier that includes at least one hydrophobic component or gelling agent and optionally water, coral-shaped metal (e.g., gold) nanoparticles dispersed throughout the carrier and forming a nanoparticle stabilizing matrix within the carrier, and spherical-shaped metal (e.g., silver) nanoparticles dispersed throughout the carrier and stabilized by the nanoparticle stabilizing matrix formed by the coral-shaped metal nanoparticles. The spherical-shaped metal nanoparticles act as a preservative and prevent spoiling of personal care products. The coral-shaped metal nanoparticles provide personal care products with hydrodynamic and antioxidant properties. The weight ratio of coral-shaped metal nanoparticles to spherical-shaped metal nanoparticles can be 1:1 to 50:1. The concentration of coral-shaped metal nanoparticles can be 1 ppm or greater, and the concentration of spherical-shaped metal nanoparticles can be less than 1 ppm. Examples of personal care products include sprays, serums, oils, gels, creams, lotions, emulsions, and semi-solids.

### CROSS-REFERENCE TO RELATED APPLICATION

This application claims the benefit of U.S. Provisional Application No. 63/324,464, filed Mar. 28, 2022, which is incorporated by reference in its entirety.

### BACKGROUND

### Technical Field

Disclosed are personal care compositions that contain antimicrobial, hydrodynamic, and antioxidant nanoparticles. Also disclosed are methods of manufacturing and using personal care compositions.

### Related Technology

Many personal care products are bought over-the-counter by customers at grocery stores and drug stores. Personal care products can often sit on a shelf for a long time before purchase and consumption. In addition, such products can often remain in warehouses or on shipping boats for a long time as they are stored and transported from manufacturing facilities to the final retail locations.

Generally, personal care products are sold for specific purposes or uses. For example, many lotions are advertised as being moisturizing for dry skin, while others are advertised as being suitable for oily skin. Some personal care products contain water or other ingredients that create a beneficial environment for the growth of bacteria, fungi and/or other microbes. Often, such growth is undesirable and can lead to spoilage of the product and a decrease in shelf life. However, customers may be unaware that the product has become spoiled prior to purchasing and/or using the product. Consumption or use of spoiled personal care products can lead to rashes, irritation, or other health problems. However, many preservatives have fallen into disfavor as being unsafe.

Conventional antimicrobial agents based on colloidal silver are ionic in nature and contain and/or release significant quantities of silver ions. Due to their ionic nature, these agents may break down and be unable to provide prolonged antimicrobial activity. Additionally, because these agents break down, a composition containing them may change in aesthetic appearance and performance over time. The breakdown of agents changes the overall composition, potentially weakening the antimicrobial or preservative nature. In addition, silver ions can be toxic to humans and animals.

Antimicrobial compositions formulated for treating skin infections and other dermatological conditions are disclosed in U.S. Pat. Nos. 9,839,863 and 10,953,043. Such antimicrobial compositions are characterized as containing a sufficiently high concentration of silver nanoparticles in order to kill microbes on or in dermal tissue and provide a therapeutic effect. Such antimicrobial compositions may optionally contain a minor quantity of gold nanoparticles to potentiate the antimicrobial activity of the silver nanoparticles. However, such antimicrobial compositions do not contain the proper types and ratios of metal nanoparticles for use in personal care compositions having desired aesthetic properties for primarily cosmetic rather than therapeutic functionalities. In addition, it was found that spherical-shaped metal nanoparticles incorporated into serums, oils, gels, lotions, creams, semi solids, or other carriers containing hydrophobic components will readily agglomerate and separate as a precipitate, apparently because of the destabilizing effects of hydrophobic components in addition to or instead of polar solvents such as water or alcohols, thus losing their beneficial antimicrobial properties.

Accordingly, there has been and remains a need to find beneficial nanoparticle systems that prevent spoiling of personal care compositions, such as cosmetics for daily or frequent skin care, while remaining stable over time.

### SUMMARY

Disclosed are embodiments of personal care (i.e., cosmetic) compositions that contain a combination of metal nanoparticles that provide desired antimicrobial, hydrodynamic, and antioxidant effects, all three of which are necessary to create long-term stability and maintain aesthetic appeal. Examples of personal care compositions include sprays (e.g., that contain at least one organic component), serums, oils, gels, lotions, creams, emulsions, and semi-solids for cosmetic uses, such as skin hydration and protection. Also disclosed are methods of manufacturing personal care compositions containing metal nanoparticles that provide desired antimicrobial, hydrodynamic, and antioxidant effects for long-term stability and aesthetic appeal

It has been found that nonionic metal nanoparticles produced by high energy methods to possess smooth morphologies devoid of crystal facets or edges and that have a narrow particle size distribution can be integrated into personal care compositions as preservatives to prevent bacterial or other microbial growth, provide an antioxidant effect to preserve color, stability, and aesthetic look, provide a hydrodynamic effect to hold significantly more water compared to conventional personal care products, and synergistically interact to prevent agglomeration and separation of metal nanoparticles from the carrier.

In some embodiments, the disclosed personal care compositions incorporate a relatively low concentration of nonionic spherical-shaped metal (e.g., silver) nanoparticles that act as a preservative to prevent or inhibit microbial growth but without having a sufficiently high concentration to provide significant therapeutic antimicrobial effects, such as treating dermal infections. The personal care compositions also contain a sufficiently high concentration of coral-shaped metal (e.g., gold) nanoparticles in order to provide three important effects: (1) create a dispersed matrix of coral-shaped metal nanoparticles within the carrier to attract and hold, but not agglomerate with, the spherical-shaped metal nanoparticles to create and maintain a high dispersion of non-agglomerated and non-precipitating metal nanoparticles; (2) hydrodynamically interact with water to yield a spray, gel, lotion, cream, other emulsion, or semi-solid having high water content while maintaining a desired stability, viscosity or semi-solid nature compared to the same composition devoid of coral-shaped metal nanoparticles; and (3) provide an antioxidant effect to preserve color, stability, and aesthetic appearance of the personal care compositions. The carrier may omit water such that the matrix-forming and antioxidant properties of coral-shaped metal nanoparticles dominate rather than hydrodynamic properties.

In some embodiments, personal care compositions comprise (1) a spray, a gel, a lotion, a cream, or a solid carrier; (2) less than 1 ppm by weight of spherical-shaped metal (e.g., silver) nanoparticles to prevent or inhibit microbial growth and spoilage; and (3) at least 1 ppm by weight of coral-shaped metal (e.g., gold) nanoparticles in a weight ratio greater than 1:1 relative to the spherical-shaped metal nanoparticles in order to create a matrix in the carrier that attracts and maintains the spherical-shaped metal nanoparticles in a dispersed, non-agglomerated, and stable condition, to provide hydrodynamic properties that permit the composition to include more water while maintaining a desired viscosity or semi-solid nature, and to provide an antioxidant effect to preserve the color, stability, and aesthetic appearance of the personal care compositions. The carrier may omit water such that the matrix-forming and antioxidant properties of coral-shaped metal nanoparticles dominate rather than hydrodynamic properties.

In some embodiments, a method of manufacturing a personal care composition comprises: (1) providing a suitable carrier base material; (2) mixing a solution or stable suspension (e.g., aqueous solution or suspension) of coral-shaped metal (e.g., gold) nanoparticles with the carrier base material to create a carrier with a matrix of well-dispersed coral-shaped metal nanoparticles; and (3) mixing spherical-shaped metal (e.g., silver) nanoparticles with the carrier containing the matrix of well-dispersed coral-shaped metal nanoparticles, wherein the coral-shaped metal nanoparticles attract but do not agglomerate with the spherical-shaped metal nanoparticles, provide hydrodynamic properties to hold more water while maintaining a desired viscosity or semi-solid nature, and provide antioxidant properties to maintain color, stability, and aesthetic appearance of the personal care compositions. The carrier may omit water such that the matrix-forming and antioxidant properties of coral-shaped metal nanoparticles dominate rather than hydrodynamic properties. The coral-shaped and spherical-shaped nanoparticles can be provided in solvents and liquids other than water, such as alcohols, organic solvents, oils, DMSO, and the like.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A-1D are transmission electron microscope (TEM) images of various non-spherical nanoparticles (i.e., that have surface edges and external bond angles) made according to conventional chemical synthesis or electrical discharge methods;

FIGS. 2A-2C are TEM images of exemplary nonionic spherical-shaped metal nanoparticles having substantially uniform size and narrow particle size distribution with no surface edges or external bond angles for use in making personal care compositions;

FIGS. 3A-3C are TEM images of nonionic coral-shaped nanoparticles for use in making personal care compositions for treating dermatological conditions;

FIGS. 4A and 4B schematically illustrated a proposed mechanism of action by which nonionic spherical-shaped nanoparticles can kill or deactivate a microbe, illustrating a microbe protein with disulfide bonds being catalytically denatured by an adjacent spherical-shaped nanoparticle;

FIG. 5 schematically illustrates a mammalian protein with disulfide bonds that are shielded so as to resist being catalytically denatured by an adjacent spherical-shaped nanoparticle; and

FIG. 6 illustrates a scanning transmission electron microscopy (STEM) image of silver nanoparticles inside a MRSA SA62 drug resistant bacterium.

### DETAILED DESCRIPTION

Disclosed are embodiments of personal care (i.e., cosmetic) compositions that contain a combination of metal nanoparticles that provide desired antimicrobial, hydrodynamic, and antioxidant effects, all three of which are necessary to create personal care products with desired properties and long-term stability. Also disclosed are methods of manufacturing the personal care compositions.

In some embodiments, spherical-shaped metal (e.g., silver) nanoparticles are included in the personal care compositions in a relatively low concentration (e.g., less than 1 ppm by weight) in order to act primarily as a preservative to protect against microbial growth and spoilage but without providing a significant therapeutic antimicrobial effect where applied.

In some embodiments, coral-shaped metal (e.g., gold) nanoparticles are included in the personal care compositions in a sufficiently high concentration (e.g., at least 1 ppm by weight) in order to create a matrix within the carrier that stabilizes the spherical-shaped metal and prevents them from agglomerating and separating from the carrier, The coral-shaped metal nanoparticles, when dispersed to form the stabilizing matrix, are capable of holding more water while maintaining a desired viscosity or semi-solid nature, resulting in compositions with higher water content compared to the same compositions devoid of the coral-shaped metal nanoparticles. The coral-shaped metal nanoparticles also provide antioxidant properties to maintain color, stability, and desired aesthetic characteristics of the personal care compositions.

As used herein, the term “personal care compositions” refers to cosmetic compositions formulated for daily use rather than therapeutically active antimicrobial compositions intended for short-term therapeutic treatment of dermal infections. Examples of personal care compositions include sprays, serums, oils, gels, lotions, creams, and semi-solid compositions. Sprays, serums, and oils can have a viscosity greater than that of water and can provide skin protection, hydration, soothing, or other beautifying effects. Gels, lotions, and creams are well-known for skin hydration, protection, and soothing. Viscous or semi-solid compositions, such as deodorant sticks, makeup, foundation, primer, concealer, lipstick, lip balm, blush, etc. can be rubbed or applied to the skin using an applicator container, stick, brush, finger, and the like. Some compositions, such as lotions and creams, can be an emulsion of water and oil, such as a water-in-oil emulsion or oil-in-water emulsion. Gels may contain water or other liquid carrier and a gelling agent. Sprays and serums may be lower viscosity emulsions or gels. Oils may contain mostly or exclusively oil as the carrier. Semi-solids can be gels, emulsions, oil-based, or wax-based, for example.

The term “nanoparticle” typically refers to particles having a largest dimension of less than 100 nm. Bulk materials typically have constant physical properties regardless of size, but at the nanoscale, size dependent properties are often observed. Thus, properties of materials change as their size approaches the nanoscale and as the percentage of atoms at the surface of a material becomes significant. For bulk materials larger than one micrometer (or micron), the percentage of atoms at the surface is insignificant in relation to the number of atoms in the bulk of the material. The interesting and sometimes unexpected properties of nanoparticles are therefore largely due to the large surface area of the material, which dominates the contributions made by the relatively small bulk of the material.

In some embodiments, the personal care compositions are applied topically to a user's skin. In some embodiments, the hydrodynamic properties provided by the coral-shaped gold nanoparticles help provide the skin with a higher ratio of water for skin hydration compared to the same composition in the absence of the coral-shaped nanoparticles. In the case of oil-based and other hydrophobic carriers, the coral-shaped nanoparticles can help lock in existing moisture within the skin. The spherical-shaped nanoparticles prevent spoilage of the personal care compositions but generally do not provide sufficient anti-microbial activity to provide a significant therapeutic antimicrobial effect (e.g., to treat infections).

### I. Metal Nanoparticles in Personal Care Compositions

Metal nanoparticles used to make personal care compositions typically include nonionic, ground state, metal nanoparticles with no external edges or bond angles that can otherwise release metal ions. Personal care compositions include a carrier, spherical-shaped metal nanoparticles in a concentration to provide a preservative effect and prevent spoilage, and coral-shaped metal nanoparticles that provide at least three functions: (1) form a matrix within the carrier that attracts but does not agglomerate with the spherical-shaped metal nanoparticles, (2) provide hydrodynamic properties that permit the inclusion of more water while maintaining a desired viscosity of semi-solid nature, and (3) provide antioxidant properties to maintain color, stability, and desired aesthetic characteristics of the personal care compositions.

The term “spherical-shaped metal nanoparticles” refers to nanoparticles that are made from one or more metals, preferably nonionic, ground state metals, having only internal bond angles and no external edges or bond angles. In this way, the spherical-shaped nanoparticles are highly resistant to ionization, highly stable, and highly resistance to agglomeration. Such nanoparticles can exhibit a high ξ-potential, which permits the spherical-shaped nanoparticles to remain dispersed within a polar solvent without a surfactant, which is a surprising and unexpected result.

In some embodiments, the spherical-shaped metal nanoparticles are discrete sphere particles having a smooth outer surface. In some instances, the outer surface is completely smooth, generally smooth, and/or somewhat smooth with no external edges or facets. The nanoparticles are beneficially smooth and thus adhere to other surfaces (i.e., surfaces of the oral cavity) by Van de Waals forces, instead of a covalent chemical bond to hard surfaces. Because of this, the nanomaterials are non-reactive with commonly used dental chemistries. Additionally, because the interaction between the nanoparticles and the target areas is a physical interaction, not a chemical one, any composition comprising the nanoparticles are not hindered when included with existing dental care formulations (e.g., toothpaste, mouth rinse, etc.) or dental care practices (e.g., fluoridation).

In some embodiments, spherical-shaped metal nanoparticles can have a diameter of about 40 nm or less, about 35 nm or less, about 30 nm or less, about 25 nm or less, about 20 nm or less, about 15 nm or less, about 10 nm or less, about 7.5 nm or less, or about 5 nm or less. Spherical-shaped metal nanoparticles preferably have a mean diameter in a range of about 1 nm to about 20 nm, more preferably in a range of about 1 nm to about 20 nm, more preferably in a range of about 2 nm to about 15 nm, and most preferably in a range of about 3 nm to about 12 nm.

In some embodiments, spherical-shaped nanoparticles can have a particle size distribution such that at least 99% of the nanoparticles have a diameter within 30% of the mean diameter of the nanoparticles, or within 20% of the mean diameter, or within 10% of the mean diameter. In some embodiments, spherical-shaped nanoparticles can have a mean particle size and at least 99% of the nanoparticles have a particle size that is within ±3 nm of the mean diameter, ±2 nm of the mean diameter, or ±1 nm of the mean diameter. In some embodiments, spherical-shaped nanoparticles can have a ξ-potential (absolute value) of at least 10 mV, preferably at least about 15 mV, more preferably at least about 20 mV, even more preferably at least about 25 mV, and most preferably at least about 30 mV.

Examples of methods and systems for manufacturing spherical-shaped metal nanoparticles by laser ablation or electric discharge to form an initial plume of nanoparticles, coupled with cross-laser manipulation of nanoparticle size, are disclosed in U.S. Pat. Nos. 9,849,512, 10,137,503, and 10,610,934 to William Niedermeyer, which are incorporated herein by reference in their entirety.

It should be understood that the spherical-shaped metal nanoparticles made according to the Niedermeyer patents differ substantially from conventional colloidal silver or other metal nanoparticles formed by chemical or other processes that yield ionic solutions and/or colloidal metal particles with crystal facets and edges that release metal ions.

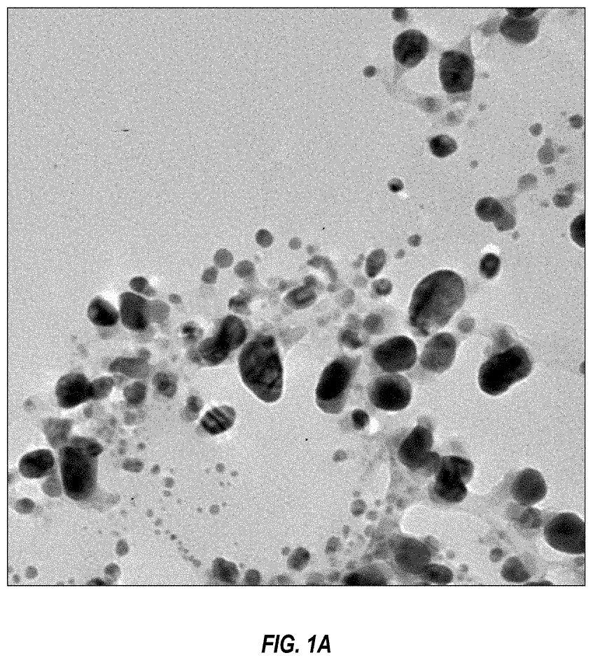

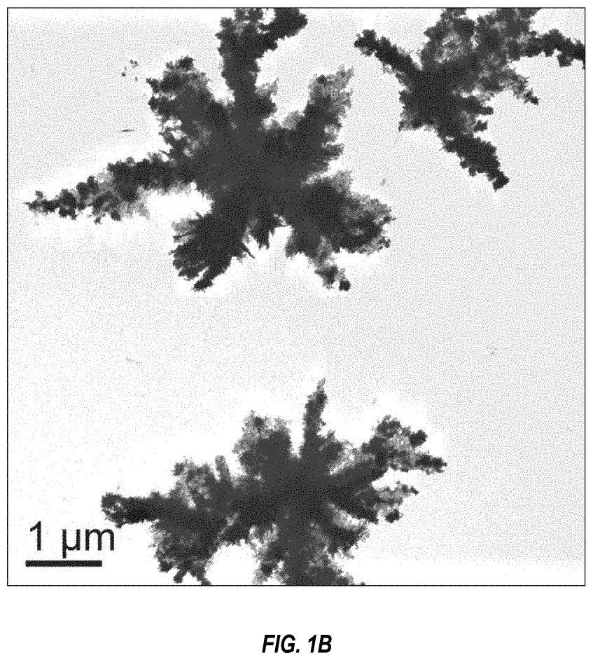

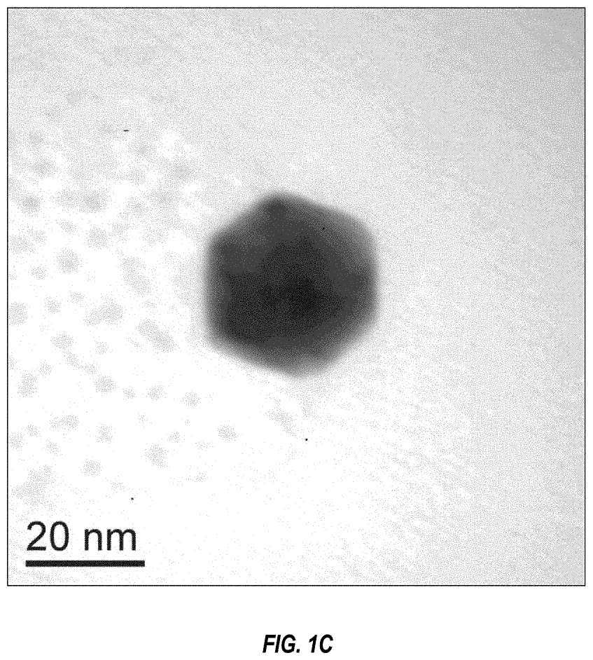

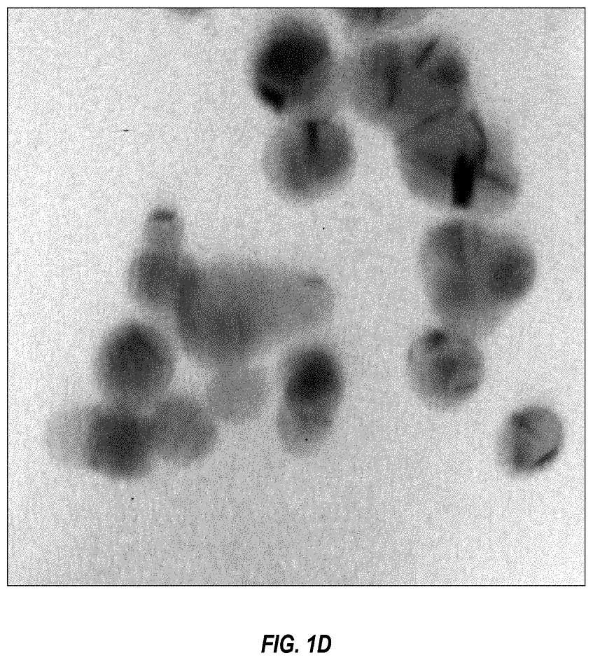

For comparison purposes, FIGS. 1A-1D are transmission electron microscope (TEM) images of metal nanoparticles made according to various chemical synthesis methods. As shown, the nanoparticles formed using these various chemical synthesis methods tend to exhibit a clustered, crystalline, faceted, or hedron-like shape rather than a true spherical shape with round and smooth surfaces.

For example, FIG. 1A shows silver nanoparticles formed using a common trisodium citrate method. The nanoparticles tend to be clustered (agglomerated) and have a broad particle size distribution (i.e., the difference in particle size between the largest particles, smallest particles, and mean particle size is very large). FIG. 1B shows another set of silver nanoparticles (available from American Biotech Labs, LLC) formed using another chemical synthesis method. Such nanoparticles have rough surface morphologies with many edges and are sometimes referred to as “nanoflowers”. They are very jagged with numerous edges or crystal facets. FIG. 1C shows a gold nanoparticle having a hedron shape (hexagonal in cross section) as opposed to having a truly spherical shape (although such particles are often called “spherical”, they are not “spherical” as that term is defined herein). FIG. 1D shows a set of silver nanoparticles (sold under the trade name MesoSilver), which have relatively smoother surface morphologies but are understood to be shells of silver formed over seeds of non-metallic core material.

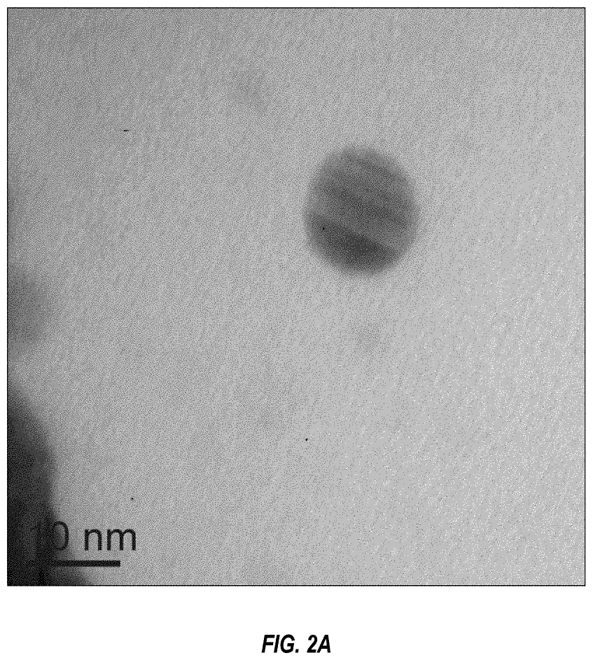

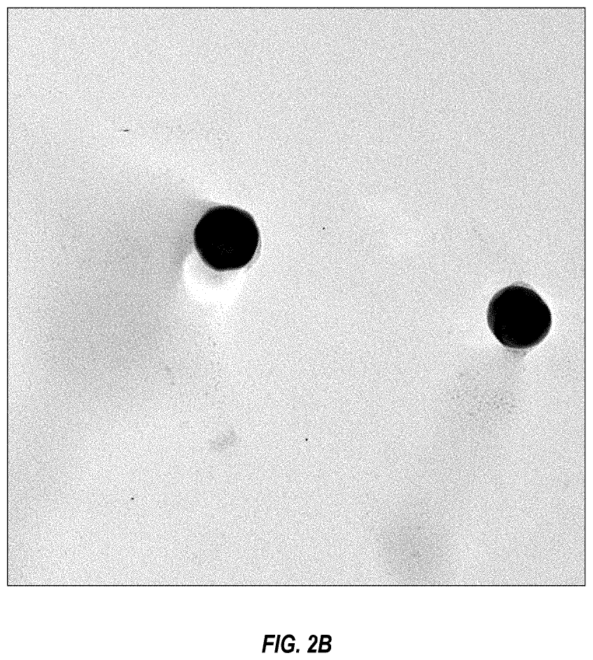

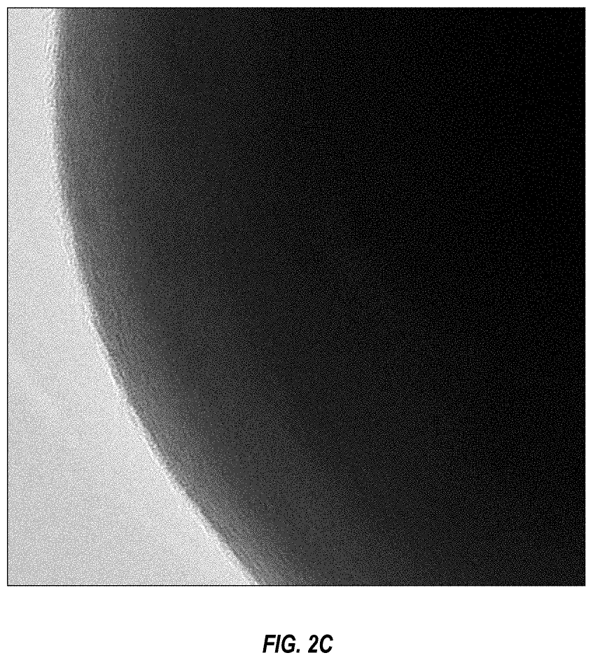

In contrast, the spherical-shaped nanoparticles described herein and incorporated into personal care compositions are nonionic, solid metal, unclustered, exposed/uncoated, and have a smooth and round surface morphology with no external bond angles or crystal facets, and have a narrow size distribution. FIGS. 2A-2C are TEM images of spherical-shaped metal nanoparticles that can be used herein. FIG. 2A shows a gold/silver alloy nanoparticle (90% silver and 10% gold by molarity). FIG. 2B shows two spherical-shaped nanoparticles side by side to visually illustrate size similarity. FIG. 2C shows a surface of a metal nanoparticle showing the smooth and edgeless surface morphology devoid of crystal facets found in conventional colloidal silver or other metal nanoparticles. The smooth surface prevents the release of metal ions compared to traditional colloidal silver, which is ionic and has external bond angles that promote ionization.

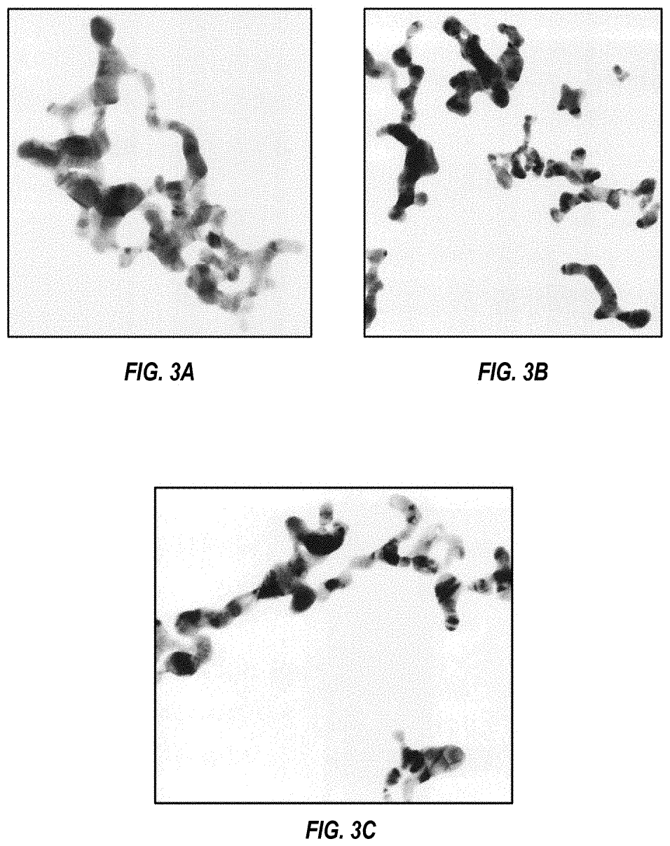

The term “coral-shaped metal nanoparticles” refers to nanoparticles that are made from one or more metals and which are nonionic and have a non-uniform cross section and globular structure formed by multiple non-linear strands joined together without right angles. Example TEM images of coral-shaped gold nanoparticles are set forth in FIGS. 3A-3C. Coral-shaped metal nanoparticles are not “nanoflowers” and have no physical or chemical resemblance to nanoflowers. Similar to spherical-shaped nanoparticles, coral-shaped nanoparticles have only internal bond angles and no external edges, crystal facets, or bond angles. In this way, coral-shaped metal nanoparticles are highly resistant to ionization, highly stable, and highly resistance to agglomeration. Also similar to spherical-shaped metal nanoparticles, coral-shaped nanoparticles can be formed as discrete particles having a smooth outer surface, and thus can achieve similar benefits as the spherical-shaped nanoparticles described above. Such coral-shaped nanoparticles can exhibit a high ξ-potential, which permits coral-shaped nanoparticles to remain dispersed within a polar solvent without a surfactant, which is a surprising and unexpected result.

In some embodiments, coral-shaped metal nanoparticles can have particle lengths ranging from about 15 nm to about 100 nm, or about 25 nm to about 95 nm, or about 40 nm to about 90 nm, or about 60 nm to about 85 nm, or about 70 nm to about 80 nm. In some embodiments, coral-shaped nanoparticles can have a particle size distribution such that at least 99% of the nanoparticles have a length within 30% of the mean length, or within 20% of the mean length, or within 10% of the mean length. In some embodiments, coral-shaped nanoparticles can have a ξ-potential of at least 10 mV, preferably at least about 15 mV, more preferably at least about 20 mV, even more preferably at least about 25 mV, and most preferably at least about 30 mV.

Examples of methods and systems for manufacturing coral-shaped metal nanoparticles by laser ablation or electric discharge to form an initial plume of nanoparticles, coupled with cross-laser manipulation of nanoparticle size, are disclosed in U.S. Pat. No. 9,919,363 to William Niedermeyer, which is incorporated by reference in its entirety. Nanoparticle compositions that contain a mixture of spherical-shaped and coral-shaped metal nanoparticles are disclosed in U.S. Pat. No. 9,434,006 to William Niedermeyer, which is incorporated by reference in its entirety.

The metal nanoparticles, including spherical-shaped and coral-shaped metal nanoparticles, may comprise any desired metal, mixture of metals, or metal alloy, including at least one of silver, gold, platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, or alloys thereof. In preferred embodiments, the spherical-shaped metal nanoparticles comprise spherical-shaped silver nanoparticles and the coral-shaped metal nanoparticles comprise coral-shaped gold nanoparticles.

It has been found that combining spherical-shaped and coral-shaped metal nanoparticles within personal care compositions, particular viscous compositions such as gels, creams, lotions, and semi-solid composition, provides unique properties and benefits due to their unique interactions within the carrier. A coral-shaped gold nanoparticle can be understood as being a dendrite with no right angles but has both protruding and inventing curved surfaces but no points or right angles. Spherical-shaped silver nanoparticle will “see” coral-shaped gold nanoparticles as having a near planar geometry with a dielectric field that is not tightly held to the surface. This is similar to the dielectric field of spherical-shaped silver nanoparticles, which is not held to the surface (i.e., is “lossy”). Both types of nanoparticles are so small that they have dielectric fields that extend from their physical particulate mass. The density difference between gold and silver increase the mass of a particle having a similar size magnitude. The nanoparticles would both move toward each other just by gravimetrics. However, silver nanoparticles move faster towards gold nanoparticles than gold nanoparticles to silver nanoparticles due to their mass differences. But because there are no right angles or points, the interaction is still seen as a planar geometry. This is physics with the thermal Brownian motion of kinetics.

There is also electrodynamics effects. If there were any right angles or facets coming together making points, or points themselves, there would be a concentration of charge at those points. This would make a non-planar geometry also a non-equal dielectric field and in the case of magnetic particles an irregular magnetic field. But gold and silver are not magnetic. Thus, the lack of irregular fields around points and edges keeps the particles from readily lining up irregular electric potentials, which would otherwise result in an agglomeration that might create a complex particle of gold and silver. However, the lack of points, facets, and edges removes this phenomenon from occurring.

Given these facts, the Brownian motion with metal nanoparticles of this size reduces the probability of collision in relation to the size and number of these nanoparticles. Only by sheer numbers that would increase the probability of collision between the two particles and the particles to each other would provide opportunity for agglomeration. However, the sizes and morphologies described above provide a factor that keeps the spherical-shaped and coral-shaped nanoparticles from agglomerating.

Thus, creating a matrix of highly dispersed coral-shaped gold nanoparticles in the carrier sets up a field of particles that are attracted to but do not agglomerate with the spherical-shaped silver nanoparticles. This is why coral-shaped gold nanoparticles are added first to the personal care compositions, followed by the spherical-shaped silver nanoparticles. If the two types of particles were put together in a single solution, there may be enough collision parameters that could cause them to agglomerate over time. However, STEM imaging has never seen this due to the current production of sustaining materials that are not agglomerative.

In general, spherical-shaped metal nanoparticles can be smaller than coral-shaped metal nanoparticles and in this way can provide very high surface area for catalyzing desired reactions or providing other desired benefits. On the other hand, the generally larger coral-shaped nanoparticles can exhibit higher surface area per unit mass compared to spherical-shaped nanoparticles because coral-shaped nanoparticles have internal spaces and surfaces rather than a solid core and only an external surface. Providing personal care compositions containing both spherical-shaped and coral-shaped metal nanoparticles can provide synergistic results. For example, coral-shaped nanoparticles can help disperse and/or potentiate the activity of spherical-shaped nanoparticles in addition to providing their own unique benefits. For example, a given concentration of spherical-shaped metal nanoparticles may offer increased antimicrobial and preservative properties due to the presence of coral-shaped metal nanoparticles. In some embodiments, a combination of spherical-shaped and coral-shaped metal nanoparticles can lead to synergistic, broad-spectrum protection against microbial spoilage at low concentration with a greater amount of protection per amount of active ingredient relative to single sized and/or shaped compositions.

In order for personal care compositions to be well-suited as cosmetic rather than therapeutically active antimicrobial compositions, the concentration of spherical-shaped metal (e.g., silver) nanoparticles is lower than in therapeutic antimicrobial compositions, such as those disclosed in U.S. Pat. Nos. 9,839,863 and 10,953,043. The concentration of spherical-shaped metal (e.g., silver) nanoparticles is selected to provide a preservative and anti-spoilage effect by preventing or inhibiting microbial growth within the personal care compositions but not so high as to provide significant therapeutic antimicrobial activity when applied to infected dermal tissue. Providing a higher concentration of spherical-shaped silver nanoparticles, such as greater than 2 ppm, or even 1 ppm or greater, can be potentially dangerous and have negative effects, particularly in a personal care composition designed for daily usage rather than for temporary treatment of a dermal infection. prolonged dosage. For example, the skin often has beneficial microbial flora, which might be killed, damaged or otherwise negatively affected through frequent application of a therapeutically effective quantity of spherical-shaped silver nanoparticles, such as at the higher concentrations contained in the dermal treatment compositions in the aforementioned patents.

On the other hand, the concentration of coral-shaped metal (e.g., gold) nanoparticles is selected to be sufficiently high to create a matrix of coral-shaped nanoparticles in the carrier to keep the spherical-shaped nanoparticles well-dispersed throughout the composition and prevent agglomeration and/or separation of spherical-shaped nanoparticles from the carrier, to provide a hydrodynamic effect to hold more water at a desired viscosity or semi-solid nature, and antioxidant properties to maintain color, stability, and aesthetic appeal.

In order to provide the desired benefits for personal care compositions that are primarily cosmetic rather than therapeutic in nature, particularly in order for the spherical-shaped metal nanoparticles to provide a preservative and anti-spoilage effect rather than a concentration effective for treat dermal infections, and to permit daily use without the potentially harmful buildup of antimicrobial silver nanoparticles, the concentration of spherical-shaped metal (e.g., silver) nanoparticles is typically less than 1 ppm (parts per million) on a weight basis, such as in a range of about 2 ppb (parts per billion) to about 900 ppb, preferably in a range of about 10 ppb to about 800 ppb, more preferably in a range of about 20 ppb to about 700 ppb, and most preferably in a range of about 30 ppb to about 600 ppb. In some embodiments, the concentration of spherical-shaped metal (e.g., silver) nanoparticles can be less than about 950 ppb, 850 ppb, 750 ppb, 650 ppb, 550 ppb, 500 ppb, 450 ppb, 400 ppb, 350 ppb, 300 ppb, 250 ppb, or 200 ppb and at least about 1 ppb, 2 ppb, 4 ppb, 7 ppb, 10 ppb, 15 ppb, 20 ppb, 25 ppb, 30 ppb, 40 ppb, 50 ppb, 60 ppb, 70 ppb, 85 ppb, or 100 ppb, or within a range with endpoints of any two of the foregoing concentrations.

With respect to coral-shaped metal nanoparticles, in order to provide the desired benefits for personal care compositions that are primarily cosmetic rather than therapeutic in nature, and which desirably have good shelf stability, both functional and aesthetic, the concentration of coral-shaped metal (e.g., gold) nanoparticles is typically at least 1 ppm (parts per million) on a weight basis, such as in a range of about 2 ppm to about 300 ppm, preferably in a range of about 3 ppm to about 200 ppm, more preferably in a range of about 4 ppm to about 150 ppm, and most preferably in a range of about 5 ppm to about 100 ppm. In some embodiments, the concentration of coral-shaped metal (e.g., gold) nanoparticles can be at least about 0.5 ppm, 0.75 ppm, 1 ppm, 1.5 ppm, 2 ppm, 2.5 ppm, 3 ppm, 4 ppm, 5 ppm, 6 ppm, 8 ppm, 10 ppm, 12 ppm, 15 ppm, 20 ppm, 25 ppm, or 30 ppm and less than about 500 ppm, 450 ppm, 400 ppm, 300 ppm, 250 ppm, 200 ppm, 175 ppm, 150 ppm, 125 ppm, 100 ppm, 80 ppm, 60 ppm, or 50 ppm, or within a range with endpoints of any two of the foregoing concentrations.

In order for the spherical-shaped and coral-shaped metal nanoparticles to provide the desired benefits for personal care compositions, as disclosed herein, the weight ratio of coral-shaped nanoparticles to spherical-shaped nanoparticles in the personal care compositions can be in a range of greater than 1:1 to about 50:1, or about 1.5:1 to about 25:1, or about 2:1 to about 15:1, or about 3:1 to about 10:1. In some embodiments, the weight ratio of coral-shaped nanoparticles to spherical-shaped nanoparticles in the personal care compositions can be at least about 0.75:1, 1:1, 1.25:1, 1.5:1, 1.75:1, 2:1, 2.5:1, 3:1, 4:1. 5:1. 6:1, 8:1, 10:1, 12:1 or 15:1 and less than about 100:1, 80:1, 60:1, 50:1, 40:1, 35:1, 30:1, 25:1 or 20:1, or within a range with endpoints of any two of the foregoing ratios.

### II. Benefits of Personal Care Compositions

In some embodiments, the personal care compositions are applied topically to a user's skin, such as arms, legs, torso, and/or face. In some embodiments, the matrix formed by the coral-shaped gold nanoparticles can help keep the spherical-shaped silver nanoparticles in place against the user's skin. By keeping the spherical-shaped silver nanoparticles in place against the user's skin, the matrix formed by the coral-shaped gold nanoparticles enables a persistent effect of gold and silver nanoparticles, which can provide a protective barrier with possibly mild antimicrobial benefits and/or hydrodynamic properties that retain water to maintain healthy, well-hydrated skin.

Thus, the coral-shaped nanoparticles disclosed are capable of forming a matrix or network when added to a carrier, such as a spray, serum, oil, gel cream, lotion, other emulsion, or semi-solid. The spherical-shaped nanoparticles may then be suspended in the matrix and remain stably dispersed throughout the carrier. By remaining suspended and dispersed within the matrix, the spherical-shaped nanoparticles are kept locally stable and available to act as a preservative. For example, the spherical-shaped metal nanoparticles can kill or deactivate microbes (described below) to prevent spoilage during storage of the personal care compositions. Beneficially, the spherical-shaped metal nanoparticles are of a size and morphology that enables them to diffuse out of a microbe after engulfment and subsequent deactivation of the microbe. After diffusing out of the dead or deactivated microbe, the spherical-shaped nanoparticles are again retained with the matrix of coral-shaped metal nanoparticles and available to kill or deactivate other microbes that may be in the composition.

This effect results in long-term stability and prevention of spoilage of the personal care compositions. The personal care compositions are advantageously shelf stable for at least about three months, six months, 9 months, 12 months, 18 months, or 24 months. Not only does long shelf stability provide economic savings, in the form of fewer damaged products due to spoilage, it also beneficially provides safer personal care compositions that are free or substantially free of microbes. Consumers of such personal care composition are unlikely to be harmed or turned off by spoiled personal care composition due to the beneficial inclusion of spherical-shaped and coral-shaped metal nanoparticles. Spoiled personal care products can cause rashes, lesions, itching, burning, or other irritations. Personal care products made from compositions containing the disclosed metal nanoparticles have a prolonged shelf-life and beneficially do not cause such irritations.

In some embodiments, the presence of the matrix of coral-shaped metal nanoparticles enables personal care compositions made thereof to have an increased water content due to their hydrodynamic properties. For example, the matrix coral-shaped metal nanoparticles can beneficially retain a higher quantity of water molecules on or withing skin where the composition is applied as compared to the same composition but devoid of the coral-shaped metal nanoparticles.

In some embodiments, the personal care compositions may include sodium laurel sulfate (SLS) at levels that are not antimicrobial. When SLS is used with spherical-shaped silver nanoparticles, it increases the antimicrobial effect of the silver nanoparticles. This is because the SLS encourages the uptake of nanoparticles associated with SLS into bacteria or other microbes. This permits for further reductions in the concentration of spherical-shaped silver nanoparticles required to provide preservative and anti-spoilage effects in the personal care compositions.

### III. Antimicrobial Activity of Spherical-Shaped Metal Nanoparticles

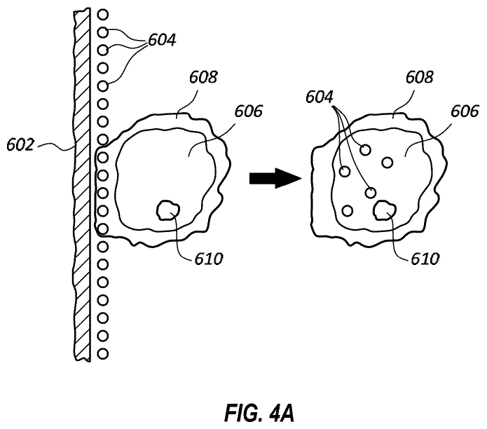

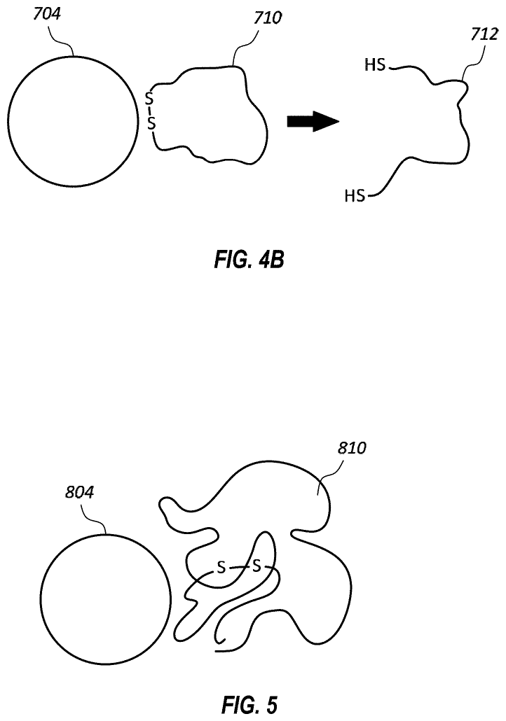

FIGS. 4A and 4B schematically illustrate a microbe after having absorbed a spherical-shaped metal nanoparticle and the subsequent denaturing of microbial proteins. FIG. 5 schematically illustrates how mammalian proteins are shielded from attack by a spherical-shaped metal nanoparticle.

FIG. 4A schematically illustrates a microbe 608 having absorbed spherical-shaped metal nanoparticles 604 from a substrate 602, such as by active absorption or other transport mechanism. It will be appreciated that spherical-shaped metal nanoparticles 604 can be provided in a composition (not shown), such as in personal care composition. The metal nanoparticles 604 can freely move throughout the interior 606 of the microbe 608 and come into contact with one or more vital proteins or enzymes 610 that, when denatured, will kill or disable the microbe.

One way that metal nanoparticles may kill or denature a microbe is by catalyzing the cleavage of disulfide (S—S) bonds within a vital protein or enzyme. FIG. 4B schematically illustrates a microbe protein or enzyme 710 with disulfide bonds being catalytically denatured by an adjacent spherical-shaped metal nanoparticle 704 to yield denatured protein or enzyme 712. In the case of bacteria or fungi, the cleavage of disulfide bonds and/or cleavage of other chemical bonds of vital proteins or enzymes may occur within the cell interior, thereby killing the microbe in this manner. Such catalytic cleavage of disulfide (S—S) or other critical chemical bonds is facilitated by the generally simple protein structures of microbes, in which many vital disulfide bonds are on exposed surfaces and readily cleaved by catalysis.

Another mechanism by which metal (e.g., silver) nanoparticles can kill microbes is through the production of active oxygen species, such as peroxides, which can oxidatively cleave protein bonds, including but not limited to amide bonds.

In the particular case of silver (Ag) nanoparticles, the interaction of the silver (Ag) nanoparticle(s) within a microbe has been demonstrated to be particularly lethal to the microbe without the release of silver ions (Ag+) to provide the desired antimicrobial effects, as is typically the case with conventional colloidal silver compositions, which are known to kill because of their high concentration of free silver ions. The ability of nonionic spherical-shaped silver nanoparticles to provide effective microbial control without significant release of toxic silver ions (Ag+) into living tissue and the environment is a substantial advancement in the art. It also renders the silver nanoparticle essentially non-toxic to animals, such as mammals, birds, reptiles, fish, and amphibians.

In the case of viruses, metal nanoparticles can alternatively deactivate viruses by attaching to glycoproteins and/or catalyzing protein denaturing reactions in the protein coat so that the virus is no longer able to attach to a host cell and/or inject genetic material into the host cell. Because very small nanoparticles can pass through a virus, denaturing of the protein coat may occur within the interior of the virus. A virus that is rendered unable to attach to a host cell and/or inject genetic material into the host cell is essentially inactive and no longer pathogenic.

Notwithstanding the lethal nature of nonionic metal nanoparticles to microbes, they are relatively harmless to humans, mammals, and healthy mammalian cells, which contain much more complex protein structures compared to simple microbes in which most or all vital disulfide bonds are shielded by other, more stable regions of the protein. FIG. 5 schematically illustrates a mammalian protein 810 with disulfide (S—S) bonds that are shielded so as to resist being catalytically denatured by an adjacent spherical-shaped nanoparticle 804. In many cases the nonionic metal nanoparticles do not interact with or attach to human or mammalian cells, remain in and follow fluid flow, do not cross barriers, remain in the vascular system, and can be quickly and safely expelled through the urine without damaging kidneys or other cells.

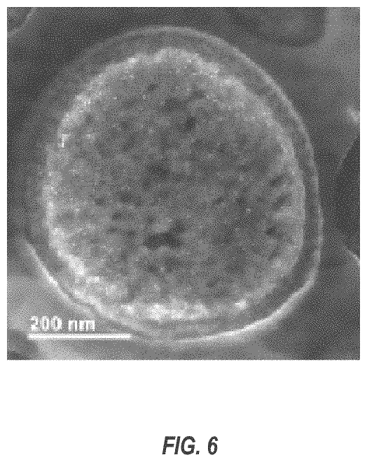

FIG. 6 is a STEM image of silver nanoparticles that have penetrated inside a drug-resistant MRSA SA62 bacterium. STEM imaging using no stain and a dark field camera with 3 nm of carbon coating allowed for tracking of the nanoparticles within and around the bacterium. The STEM imaging in coordination with Electron Diffraction Spectroscopy (EDS) provided confirmation of sulfur stripping from the exposed site of disulfide bonds and ferredoxins.

### IV. Carriers for Personal Care Products

In some embodiments, the spherical and/or coral-shaped metal nanoparticles are added to one or more carriers and/or stabilizing agents. Examples of stabilizing agents include deionized water and alcohol (e.g., ethanol), as deionized water and alcohols have been observed to effectively maintain nanoparticles of different sizes and different shapes within a given solution.

Given the ability of stabilizing agents to readily dissolve into carrier materials of personal care compositions, such as water, alcohols and/or oils, introduction or manufacture of the nanoparticles into a solution with a stabilizing agent allows the nanoparticles to be readily incorporated into any number of carriers that may then become the basis for a wide array of personal care compositions, including sprays, serums, oils, gels, lotions, creams, emulsions, or semi-solid cosmetics. In some embodiments, the nanoparticles may be incorporated into a carrier that is a cosmetic composition or may be incorporated into an ingredient that is included in a cosmetic composition. For example, nanoparticle compositions may be incorporated into a carrier that forms or is part of makeup (e.g., primer, concealer, foundation, powder, blush, etc.), lipstick, lip balm, and the like.

In some embodiments, the carrier is an emulsion, such as a water in oil emulsion or an oil in water emulsion, to form a cream, lotion, or other emulsion. The oil portion may comprise at least one of one or more plant-based oils, one or more animal-based oils, one or more fatty acids, or one or more mineral oils. The emulsion may further contain an emulsifier and optionally one or more of a thickener, preservative, fragrance, colorant, or other component(s) commonly contained in creams, lotions, or other emulsions.

In some embodiments, the carrier is a gel, which typically includes primarily water, a gelling agent, and optionally one or more of a preservative, fragrance, colorant, or other component(s) commonly contained in gels. Example gelling agent include, but are not limited to, gellan gum, pectin, agar, carrageenan, xanthan gum, alginate, starch, modified starch, gum Arabic, guar gum, locust bean gum, konjac, gum tragacanth, acacia gum, gum karaya, methyl cellulose, carboxymethyl cellulose, hydroxypropylmethyl cellulose, agar, pullulan, konjac, other cellulosic compounds, other polysaccharides, carbomer, gelatin, collagen, casein, other proteins, and silica fume.

In some embodiments, the carrier is a semi-viscous liquid spray, which may be an emulsion, gel, or other material known in the art of semi-viscous liquid sprays.

In some embodiments, the carrier is a serum, which can be water-based or oil-based but is not an emulsion or gel and can be formulated according to known methods for making cosmetic serums.

In some embodiments, the carrier comprises one or more oils and optionally one or more of a thickener, preservative, fragrance, colorant, or other component(s) commonly contained cosmetic oils. Example oils include one or more plant-based oils, one or more animal-based oils, one or more fatty acids, and one or more mineral oils.

Examples of other carriers include organic solvents and DMSO.

In some embodiments, the carrier is a semi-solid cosmetic, such as a deodorant stick, lipstick, lip balm, foundation, blush, and the like and can be formulated according to known methods for making semi-solid cosmetics. In some embodiments, the semi-solid cosmetic comprises a highly viscous oil component and/or a wax component.

Other examples of carriers, stabilizing agents, and optional components are disclosed in U.S. Pat. Nos. 9,839,863 and 10,953,043, which are incorporated by reference in their entirety.

It has been found that gel carriers, emulsions, oils, and carriers that contain significant quantities of hydrophobic materials do not reliably maintain dispersions of spherical-shaped metal nanoparticles. Rather, carriers other than water, alcohols, or other hydrophilic solvents cause or allow spherical-shaped metal nanoparticles to agglomerate and precipitate out as solid particles. It was unexpectedly found that also including coral-shaped metal nanoparticles in sufficiently high concentrations relative to the carrier and/or spherical-shaped metal nanoparticles form a matrix or web that is attractive but not agglomerative to spherical-shaped metal nanoparticles. In this way, forming a carrier that first includes a matrix of dispersed coral-shaped metal nanoparticles and then adding spherical-shaped metal nanoparticles to this material prevents or inhibits agglomeration and precipitation of spherical-shaped metal nanoparticles from the carrier.

### V. Method of Manufacturing Personal Care Compositions

Also disclosed are methods of manufacturing personal care compositions. In some embodiments, a method of manufacturing a personal care composition comprises: (1) providing a suitable carrier base material; (2) mixing a solution or stable suspension (e.g., aqueous solution or suspension) of coral-shaped metal (e.g., gold) nanoparticles with the carrier base material to create a carrier with a matrix of well-dispersed coral-shaped metal nanoparticles; and (3) mixing spherical-shaped metal (e.g., silver) nanoparticles with the carrier containing the matrix of well-dispersed coral-shaped metal nanoparticles, wherein the coral-shaped metal nanoparticles attract but do not agglomerate with the spherical-shaped metal nanoparticles, provide hydrodynamic properties to hold more water while maintaining a desired viscosity or semi-solid nature, and provide antioxidant properties to maintain color, stability, and aesthetic appearance of the personal care compositions. The coral-shaped and spherical-shaped nanoparticles can be provided in solvents and liquids other than water, such as alcohols, organic solvents, oils, DMSO, and the like.

In some embodiments, a method of manufacturing personal care compositions includes adding coral-shaped metal (e.g., gold) nanoparticles to a carrier base material, such as using a solution of suspension of coral-shaped metal nanoparticles, to form a carrier containing a matrix of coral-shaped metal nanoparticles. In some embodiments, the carrier is a dermatologically appropriate carrier. Thereafter, spherical-shaped metal (e.g., silver) nanoparticles are added to the carrier, wherein the matrix of coral-shaped metal nanoparticles attracts the spherical-shaped metal nanoparticles and keeps them well-dispersed throughout the carrier of the personal care composition without agglomeration, precipitation, or separation from the carrier.

It has been found that the order of adding the metal nanoparticles to the carrier is important. Thus, the coral-shaped metal nanoparticles are advantageously added to the carrier base material before adding the spherical-shaped metal nanoparticles to form a matrix of coral-shaped metal nanoparticles that subsequently attracts but does not does not agglomerate with the spherical-shaped metal nanoparticles. If instead, the spherical-shaped metal nanoparticles are added first, there is a possibility that oils within the carrier may cause agglomeration, precipitation or separation of the nanoparticles. Depending on the carrier, particularly those which contain significant to substantial quantities of oils or other hydrophobic materials, such agglomeration, precipitation or separation of spherical-shaped metal nanoparticles may be irreversible, or at least difficult to reverse, by subsequent addition of the coral-shaped metal nanoparticles.

For example, in some embodiments the spherical-shaped nanoparticles are added to the carrier after the coral-shaped metal nanoparticles have been added to form a matrix of such nanoparticles. In some embodiments, spherical-shaped silver nanoparticles are added to the carrier after the coral-shaped gold nanoparticles have been added. In such embodiments, the gold nanoparticles attract but do not agglomerate with the silver nanoparticles. The gold nanoparticles instead create a matrix that attracts and loosely maintains the silver nanoparticles within it.

In other words, adding the coral-shaped gold nanoparticles to the carrier first produces a field or matrix of nanoparticles that are attractive, but beneficially not agglomerative, to the later-added spherical-shaped silver nanoparticles. Thus, the spherical-shaped silver nanoparticles are advantageously added to the carrier after adding the coral-shaped gold nanoparticles. If the coral-shaped and spherical-shaped nanoparticles are simply mixed together in a single solution, there may be enough collision parameters present that can cause the two types of nanoparticles to agglomerate together. This effect can become more pronounced over time. Such agglomeration would reduce the preservative and anti-spoilage effect of the spherical-shaped silver nanoparticles, potentially leading to spoilage of any personal care or other product. Agglomeration may also significantly or substantially reduce the hydrodynamic and antioxidant properties of the coral-shaped gold nanoparticles.

Beneficially, the size and morphologies of the individual nanoparticles disclosed (spherical and coral-shaped), produce nanoparticles that are not agglomerative to each other. STEM imaging has confirmed this interactivity (or lack thereof), as agglomeration between gold, coral-shaped and silver, spherical-shaped nanoparticles has not been seen. The agglomerative effect is imaged by dehydrating gold coral-shaped with silver spherical-shaped nanoparticles, causing a hyper concentration between the two. Then, the dehydrated nanoparticles are resuspended in a liquid, applied to a STEM grid and imaged on the STEM to show the agglomerative behavior. However, this behavior has not been seen using such STEM imaging techniques.

### VI. Examples

### Comparative Example 1

Stable antimicrobial nanoparticle solutions for treating a tissue disease, infection or condition included a hydrophilic liquid carrier composed of water and isopropyl alcohol. Spherical-shaped silver nanoparticles were dispersed in the hydrophilic liquid carrier at various concentrations from 1 to 5 ppm. The hydrophilic liquid carrier maintained stable dispersions of the spherical-shaped silver nanoparticles because of the high ξ-potential of the nanoparticles. The stable antimicrobial nanoparticle solutions were effective in treating skin and other infections when applied thereto.

### Comparative Example 2

Antimicrobial nanoparticle gel compositions were made by dispersing spherical-shaped silver nanoparticles at various concentrations from 1 to 5 ppm into gel carriers made using water and a gelling agent such as carbomer or xanthan gum. The nanoparticle gel compositions were found to be unstable over time, with the spherical-shaped silver nanoparticles agglomerating and precipitating out of the gel carrier within a few weeks or months. This destroyed the antimicrobial properties that the spherical-shaped silver nanoparticles possessed when initially dispersed in the gel carrier.

### Comparative Example 3

Antimicrobial nanoparticle creams and lotions were made by dispersing spherical-shaped silver nanoparticles at various concentrations from 1 to 5 ppm into cream and lotion carriers made using water, an oil, and an emulsifier to form an emulsion. The nanoparticle creams and lotions were found to be unstable over time, with the spherical-shaped silver nanoparticles agglomerating and precipitating out of the cream and lotion carriers within a few weeks or months. This destroyed the antimicrobial properties that the spherical-shaped silver nanoparticles possessed when initially dispersed in the cream and lotion carriers.

### Comparative Example 4

Antimicrobial nanoparticle oil compositions are made by dispersing spherical-shaped silver nanoparticles at various concentrations from 1 to 5 ppm into an oil carrier. The nanoparticle oil compositions are unstable over time, with the spherical-shaped silver nanoparticles agglomerating and precipitating out of the oil carrier within days, weeks or months. This destroyed the antimicrobial properties that the spherical-shaped silver nanoparticles would otherwise possess when initially dispersed in the oil carrier.

### Comparative Example 5

Antimicrobial nanoparticle semi-solid nanoparticle cosmetic compositions are made by dispersing spherical-shaped silver nanoparticles at various concentrations from 1 to 5 ppm into a semi-solid carrier comprising at least one of a highly viscous oil or wax. The semi-solid nanoparticle cosmetic compositions are unstable over time, with the spherical-shaped silver nanoparticles agglomerating and precipitating out of the semi-solid carrier within days, weeks or months. This destroyed the antimicrobial properties that the spherical-shaped silver nanoparticles would otherwise possess when initially dispersed in the semi-solid carrier.

### Example 1

Cosmetic nanoparticle gel compositions were made by initially dispersing coral-shaped gold nanoparticles at various concentrations from 1 to 5 ppm into gel carriers made using water and a gelling agent such as carbomer or xanthan gum to form stabilized gel carriers comprising a matrix of well-dispersed coral-shaped gold nanoparticles. Spherical-shaped silver nanoparticles were mixed into the stabilized gel carriers at various concentrations below 1 ppm, including concentrations between 100 ppb and 800 ppb. The coral-shaped gold nanoparticles had a mean length of 80 nm, and 99% of the nanoparticles had a length within ±10 nm of the mean length. The spherical-shaped silver nanoparticles had a mean diameter of 10 nm, and 99% of the nanoparticles had a diameter within ±1 nm of the mean diameter. The weight ratio of coral-shaped gold nanoparticles to spherical-shaped silver nanoparticles was greater than 1:1 and up to about 10:1.

The cosmetic nanoparticle gel compositions were found to remain stable for at least 12-24 months, with the spherical-shaped silver nanoparticles remaining well-dispersed and non-agglomerated within the gel carriers. The spherical-shaped silver nanoparticles acted as a preservative and prevented microbial growth and spoilage of the cosmetic nanoparticle gel compositions. The coral-shaped gold nanoparticles provided additional stability by keeping the spherical-shaped silver nanoparticles well dispersed and by providing antioxidant properties. In addition, the coral-shaped gold nanoparticles provided hydrodynamic properties that permitted the cosmetic nanoparticle gel compositions to have greater viscosity at a given water concentration. Stated another way, the coral-shaped gold nanoparticles permitted the cosmetic nanoparticle gel compositions to incorporate more water while maintaining a desired viscosity compared to the same gel compositions devoid of the coral-shaped gold nanoparticles.

### Example 2

Cosmetic nanoparticle cream and lotion compositions were made by initially dispersing coral-shaped gold nanoparticles at various concentrations from 1 to 5 ppm into cream and lotion base carriers made using water, oil, and an emulsifier to form stabilized cream and lotion carriers comprising a matrix of well-dispersed coral-shaped gold nanoparticles. Spherical-shaped silver nanoparticles were mixed into the stabilized cream and lotion carriers at various concentrations below 1 ppm, including concentrations between 100 ppb and 800 ppb. The coral-shaped gold nanoparticles had a mean length of 80 nm, and 99% of the nanoparticles had a length within ±10 nm of the mean length. The spherical-shaped silver nanoparticles had a mean diameter of 10 nm, and 99% of the nanoparticles had a diameter within ±1 nm of the mean diameter. The weight ratio of coral-shaped gold nanoparticles to spherical-shaped silver nanoparticles was greater than 1:1 and up to about 10:1.

The cosmetic nanoparticle cream and lotion compositions were found to remain stable for at least 12-24 months, with the spherical-shaped silver nanoparticles remaining well-dispersed and non-agglomerated within the cream and lotion carriers. The spherical-shaped silver nanoparticles acted as a preservative and prevented microbial growth and spoilage of the cosmetic nanoparticle cream and lotion compositions. The coral-shaped gold nanoparticles provided additional stability by keeping the spherical-shaped silver nanoparticles well dispersed and by providing antioxidant properties. In addition, the coral-shaped gold nanoparticles provided hydrodynamic properties that permitted the cosmetic nanoparticle cream and lotion compositions to have greater viscosity at a given water concentration. Stated another way, the coral-shaped gold nanoparticles permitted the cosmetic nanoparticle cream and lotion compositions to incorporate more water while maintaining a desired viscosity compared to the same cream and lotion compositions devoid of the coral-shaped gold nanoparticles.

### Example 3

Cosmetic nanoparticle gel compositions are made by initially dispersing coral-shaped gold nanoparticles at various concentrations from 1 to 5 ppm into a gel base carrier containing water, ethyl alcohol, sodium hydroxide, carbopol, and sodium hyaluronate to form stabilized gel carriers comprising a matrix of well-dispersed coral-shaped gold nanoparticles. Spherical-shaped silver nanoparticles are mixed into the stabilized gel carriers at various concentrations below 1 ppm, including concentrations between 100 ppb and 800 ppb. The coral-shaped gold nanoparticles have a mean length of 80 nm, and 99% of the nanoparticles have a length within ±10 nm of the mean length. The spherical-shaped silver nanoparticles have a mean diameter of 10 nm, and 99% of the nanoparticles have a diameter within ±1 nm of the mean diameter. The weight ratio of coral-shaped gold nanoparticles to spherical-shaped silver nanoparticles is greater than 1:1 and up to about 10:1.

The cosmetic nanoparticle gel compositions remain stable for at least 12-24 months, with the spherical-shaped silver nanoparticles remaining well-dispersed and non-agglomerated within the gel carriers. The spherical-shaped silver nanoparticles act as a preservative and prevent microbial growth and spoilage of the cosmetic nanoparticle gel compositions. The coral-shaped gold nanoparticles provide additional stability by keeping the spherical-shaped silver nanoparticles well dispersed and by providing antioxidant properties. In addition, the coral-shaped gold nanoparticles provide hydrodynamic properties that permit the cosmetic nanoparticle gel compositions to have greater viscosity at a given water concentration. Stated another way, the coral-shaped gold nanoparticles permit the cosmetic nanoparticle gel compositions to incorporate more water while maintaining a desired viscosity compared to the same gel compositions devoid of the coral-shaped gold nanoparticles.

### Example 4

Cosmetic nanoparticle lotion compositions are made by initially dispersing coral-shaped gold nanoparticles at various concentrations from 1 to 5 ppm into a lotion base carrier made using water, glycerin, coco caprylate, caprylic capric triglycerides, cetearyl alcohol, glyceryl stearate, sodium hyaluronate, hydrogenated olive oil, sodium stearoyl glutamate, olive fruit oil, xanthan gum, olive oil unsaponifiables, mixed tocopherol, sodium polyacrylate, caprylyl glycol, and ethylhexyl glycerin to form stabilized lotion carriers comprising a matrix of well-dispersed coral-shaped gold nanoparticles. Spherical-shaped silver nanoparticles are mixed into the stabilized lotion carriers at various concentrations below 1 ppm, including concentrations between 100 ppb and 800 ppb. The coral-shaped gold nanoparticles have a mean length of 80 nm, and 99% of the nanoparticles have a length within ±10 nm of the mean length. The spherical-shaped silver nanoparticles have a mean diameter of 10 nm, and 99% of the nanoparticles have a diameter within ±1 nm of the mean diameter. The weight ratio of coral-shaped gold nanoparticles to spherical-shaped silver nanoparticles is greater than 1:1 and up to about 10:1.

The cosmetic nanoparticle lotion compositions remain stable for at least 12-24 months, with the spherical-shaped silver nanoparticles remaining well-dispersed and non-agglomerated within the lotion carriers. The spherical-shaped silver nanoparticles act as a preservative and prevent microbial growth and spoilage of the cosmetic nanoparticle lotion compositions. The coral-shaped gold nanoparticles provide additional stability by keeping the spherical-shaped silver nanoparticles well dispersed and by providing antioxidant properties. In addition, the coral-shaped gold nanoparticles provide hydrodynamic properties that permit the cosmetic nanoparticle lotion compositions to have greater viscosity at a given water concentration. Stated another way, the coral-shaped gold nanoparticles permit the cosmetic nanoparticle lotion compositions to incorporate more water while maintaining a desired viscosity compared to the same lotion compositions devoid of the coral-shaped gold nanoparticles.

### Example 5

Cosmetic nanoparticle oil compositions are made by initially dispersing coral-shaped gold nanoparticles at various concentrations from 1 to 5 ppm into oil base carriers containing one or more oil components to form stabilized oil carriers comprising a matrix of well-dispersed coral-shaped gold nanoparticles. Spherical-shaped silver nanoparticles are mixed into the stabilized oil carriers at various concentrations below 1 ppm, including concentrations between 100 ppb and 800 ppb. The coral-shaped gold nanoparticles have a mean length of 80 nm, and 99% of the nanoparticles had a length within ±10 nm of the mean length. The spherical-shaped silver nanoparticles have a mean diameter of 10 nm, and 99% of the nanoparticles had a diameter within ±1 nm of the mean diameter. The weight ratio of coral-shaped gold nanoparticles to spherical-shaped silver nanoparticles is greater than 1:1 and up to about 10:1.

The cosmetic nanoparticle oil compositions remain stable for at least 12-24 months, with the spherical-shaped silver nanoparticles remaining well-dispersed and non-agglomerated within the oil carriers. The spherical-shaped silver nanoparticles act as a preservative and prevent microbial growth and spoilage of the cosmetic nanoparticle oil compositions. The coral-shaped gold nanoparticles provide additional stability by keeping the spherical-shaped silver nanoparticles well dispersed and by providing antioxidant properties.

### Example 6

Semi-solid nanoparticle cosmetic compositions are made by initially dispersing coral-shaped gold nanoparticles at various concentrations from 1 to 5 ppm into semi-solid base carriers comprising at least one of a highly viscous oil or wax to form stabilized semi-solid carriers comprising a matrix of well-dispersed coral-shaped gold nanoparticles. Spherical-shaped silver nanoparticles are mixed into the stabilized semi-solid carriers at various concentrations below 1 ppm, including concentrations between 100 ppb and 800 ppb. The coral-shaped gold nanoparticles have a mean length of 80 nm, and 99% of the nanoparticles had a length within ±10 nm of the mean length. The spherical-shaped silver nanoparticles have a mean diameter of 10 nm, and 99% of the nanoparticles had a diameter within ±1 nm of the mean diameter. The weight ratio of coral-shaped gold nanoparticles to spherical-shaped silver nanoparticles is greater than 1:1 and up to about 10:1.

The semi-solid nanoparticle cosmetic compositions remain stable for at least 12-24 months, with the spherical-shaped silver nanoparticles remaining well-dispersed and non-agglomerated within the semi-solid carriers. The spherical-shaped silver nanoparticles act as a preservative and prevent microbial growth and spoilage of the semi-solid nanoparticle cosmetic compositions. The coral-shaped gold nanoparticles provide additional stability by keeping the spherical-shaped silver nanoparticles well dispersed and by providing antioxidant properties. The semi-solid nanoparticle cosmetic compositions are in various forms, including lipstick, lip balm, foundation, and blush.

### Example 7

Cosmetic nanoparticle serum compositions are made by initially dispersing coral-shaped gold nanoparticles at various concentrations from 1 to 5 ppm into a serum base carrier containing water, glycerin, hyaluronic acid (optionally neutralized as sodium hyaluronate), citric acid, polysorbate 20, potassium sorbate, and benzoic acid to form a stabilized serum carrier comprising a matrix of well-dispersed coral-shaped gold nanoparticles. Spherical-shaped silver nanoparticles are mixed into the stabilized serum carrier at various concentrations below 1 ppm, including concentrations between 100 ppb and 800 ppb. The coral-shaped gold nanoparticles have a mean length of 80 nm, and 99% of the nanoparticles had a length within ±10 nm of the mean length. The spherical-shaped silver nanoparticles have a mean diameter of 10 nm, and 99% of the nanoparticles had a diameter within ±1 nm of the mean diameter. The weight ratio of coral-shaped gold nanoparticles to spherical-shaped silver nanoparticles is greater than 1:1 and up to about 10:1.

The cosmetic nanoparticle serum compositions remain stable for at least 12-24 months, with the spherical-shaped silver nanoparticles remaining well-dispersed and non-agglomerated within the serum carrier. The spherical-shaped silver nanoparticles act as a preservative and prevent microbial growth and spoilage of the cosmetic nanoparticle serum compositions. The coral-shaped gold nanoparticles provide additional stability by keeping the spherical-shaped silver nanoparticles well dispersed and by providing antioxidant properties. In addition, the coral-shaped gold nanoparticles provide hydrodynamic properties that permit the cosmetic nanoparticle serum compositions to have greater viscosity at a given water concentration. Stated another way, the coral-shaped gold nanoparticles permit the cosmetic nanoparticle serum compositions to incorporate more water while maintaining a desired viscosity compared to the same cosmetic serum composition devoid of the coral-shaped gold nanoparticles.

### Example 8

Cosmetic nanoparticle spray compositions are made by initially dispersing coral-shaped gold nanoparticles at various concentrations from 1 to 5 ppm into a cosmetic spray base carrier containing water, butylene glycol, betaine, PPG, PEG, castor oil, hydroxypropyl cyclodextrin, silica, mica, pentylene glycol, disodium phosphate, tin oxide, sodium phosphate, benzyl alcohol, sodium benzoate, potassium sorbate, parfum (fragrance), and titanium dioxide to form a stabilized spray carrier comprising a matrix of well-dispersed coral-shaped gold nanoparticles. Spherical-shaped silver nanoparticles are mixed into the stabilized spray carrier at various concentrations below 1 ppm, including concentrations between 100 ppb and 800 ppb. The coral-shaped gold nanoparticles have a mean length of 80 nm, and 99% of the nanoparticles had a length within ±10 nm of the mean length. The spherical-shaped silver nanoparticles have a mean diameter of 10 nm, and 99% of the nanoparticles had a diameter within ±1 nm of the mean diameter. The weight ratio of coral-shaped gold nanoparticles to spherical-shaped silver nanoparticles is greater than 1:1 and up to about 10:1.

The cosmetic nanoparticle spray compositions remain stable for at least 12-24 months, with the spherical-shaped silver nanoparticles remaining well-dispersed and non-agglomerated within the spray carrier. The spherical-shaped silver nanoparticles act as a preservative and prevent microbial growth and spoilage of the cosmetic nanoparticle spray compositions. The coral-shaped gold nanoparticles provide additional stability by keeping the spherical-shaped silver nanoparticles well dispersed and by providing antioxidant properties. In addition, the coral-shaped gold nanoparticles provide hydrodynamic properties that permit the cosmetic nanoparticle spray compositions to have greater viscosity at a given water concentration. Stated another way, the coral-shaped gold nanoparticles permit the cosmetic nanoparticle spray compositions to incorporate more water while maintaining a desired viscosity compared to the same cosmetic spray composition devoid of the coral-shaped gold nanoparticles.

### Example 9

Cosmetic nanoparticle cream compositions are made by initially dispersing coral-shaped gold nanoparticles at various concentrations from 1 to 5 ppm into a cream base carrier made by heating stearic acid, olive oil and/or coconut oil, and emulsifying wax to between 160 and 200° F. and then cooling the composition to about 105° F. to form stabilized cream carriers comprising a matrix of well-dispersed coral-shaped gold nanoparticles. Spherical-shaped silver nanoparticles are mixed into the stabilized cream carrier at various concentrations below 1 ppm, including concentrations between 100 ppb and 800 ppb. The coral-shaped gold nanoparticles have a mean length of 80 nm, and 99% of the nanoparticles had a length within ±10 nm of the mean length. The spherical-shaped silver nanoparticles have a mean diameter of 10 nm, and 99% of the nanoparticles had a diameter within ±1 nm of the mean diameter. The weight ratio of coral-shaped gold nanoparticles to spherical-shaped silver nanoparticles is greater than 1:1 and up to about 10:1.

The cosmetic nanoparticle cream compositions remain stable for at least 12-24 months, with the spherical-shaped silver nanoparticles remaining well-dispersed and non-agglomerated within the cream carriers. The spherical-shaped silver nanoparticles act as a preservative and prevent microbial growth and spoilage of the cosmetic nanoparticle cream compositions. The coral-shaped gold nanoparticles provide additional stability by keeping the spherical-shaped silver nanoparticles well dispersed and by providing antioxidant properties.

### Example 10

The cosmetic nanoparticle compositions of any of Examples 1-8 are modified by substituting at least a portion of the coral-shaped gold nanoparticles with coral-shaped metal nanoparticles made using one or more other metals selected from platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, alloys of silver and gold, heterogeneous mixtures thereof, or alloys thereof. The cosmetic nanoparticle compositions remain stable for at least 6 months.

### Example 11

The cosmetic nanoparticle compositions of any of Examples 1-10 are modified by substituting at least a portion of the spherical-shaped silver nanoparticles with spherical-shaped metal nanoparticles made using one or more other metals selected from gold, platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, alloys of silver and gold, heterogeneous mixtures thereof, or alloys thereof. The cosmetic nanoparticle compositions remain stable for at least 6 months.

### Example 12

The cosmetic nanoparticle compositions of any of Examples 1-11 are modified by using different coral-shaped gold nanoparticles having mean lengths of 40 nm, 50 nm, 60 nm, 70 nm, 90 nm, and 100 nm, respectively. The cosmetic nanoparticle compositions remain stable for at least 6 months.

### Example 13

The cosmetic nanoparticle compositions of any of Examples 1-12 are modified by using different spherical-shaped silver nanoparticles having mean diameters of 4 nm, 5 nm, 6 nm, 7 nm, 8 nm, 12 nm, or 15 nm, respectively. The cosmetic nanoparticle compositions remain stable for at least 6 months.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A personal care composition, comprising:
a carrier comprised of at least one hydrophobic component or gelling agent and optionally water;
coral-shaped metal nanoparticles dispersed throughout the carrier and forming a nanoparticle stabilizing matrix within the carrier; and
spherical-shaped metal nanoparticles dispersed throughout the carrier and stabilized by the nanoparticle stabilizing matrix formed by the coral-shaped metal nanoparticles.

2. The personal care composition of claim 1, wherein the personal care composition has a weight ratio of coral-shaped metal nanoparticles to spherical-shaped metal nanoparticles of greater than 1:1.

3. The personal care composition of claim 2, wherein the weight ratio of coral-shaped metal nanoparticles to spherical-shaped metal nanoparticles is greater than 1:1 to about 50:1, or about 1.5:1 to about 25:1, or about 2:1 to about 15:1, or about 3:1 to about 10:1.

4. The personal care composition of claim 1, wherein a weight ratio of coral-shaped nanoparticles to spherical-shaped nanoparticles in the personal care composition is at least about 0.75:1, 1:1, 1.25:1, 1.5:1, 1.75:1, 2:1, 2.5:1, 3:1, 4:1. 5:1. 6:1, 8:1, 10:1, 12:1 or 15:1 and less than about 100:1, 80:1, 60:1, 50:1, 40:1, 35:1, 30:1, 25:1 or 20:1, or within a range with endpoints of any two of the foregoing ratios.

5. The personal care composition of claim 1, wherein the personal care composition includes less than 1 ppm (parts per million) of spherical-shaped metal nanoparticles on a weight basis.

6. The personal care composition of claim 5, wherein the personal care composition includes spherical-shaped metal nanoparticles at a concentration in a range from about 2 ppb (parts per billion) to about 900 ppb, or from about 10 ppb to about 800 ppb, or from about 20 ppb to about 700 ppb, or from about 30 ppb to about 600 ppb.

7. The personal care composition of claim 5, wherein the concentration of spherical-shaped metal nanoparticles is less than about 950 ppb, 850 ppb, 750 ppb, 650 ppb, 550 ppb, 500 ppb, 450 ppb, 400 ppb, 350 ppb, 300 ppb, 250 ppb, or 200 ppb and at least about 1 ppb, 2 ppb, 4 ppb, 7 ppb, 10 ppb, 15 ppb, 20 ppb, 25 ppb, 30 ppb, 40 ppb, 50 ppb, 60 ppb, 70 ppb, 85 ppb, or 100 ppb, or within a range with endpoints of any two of the foregoing concentrations.

8. The personal care composition of claim 1, wherein the personal care composition includes at least 1 ppm of coral-shaped metal nanoparticles on a weight basis.

9. The personal care composition of claim 8, wherein the personal care composition includes coral-shaped metal nanoparticles at a concentration in a range from about 2 ppm to about 300 ppm, or about 3 ppm to about 200 ppm, or about 4 ppm to about 150 ppm, or about 5 ppm to about 100 ppm.

10. The personal care composition of claim 1, wherein the personal care composition includes coral-shaped metal nanoparticles at a concentration of at least about 0.5 ppm, 0.75 ppm, 1 ppm, 1.5 ppm, 2 ppm, 2.5 ppm, 3 ppm, 4 ppm, 5 ppm, 6 ppm, 8 ppm, 10 ppm, 12 ppm, 15 ppm, 20 ppm, 25 ppm, or 30 ppm and less than about 500 ppm, 450 ppm, 400 ppm, 300 ppm, 250 ppm, 200 ppm, 175 ppm, 150 ppm, 125 ppm, 100 ppm, 80 ppm, 60 ppm, or 50 ppm, or within a range with endpoints of any two of the foregoing concentrations.

11. The personal care composition of claim 1, wherein the spherical-shaped metal nanoparticles comprise spherical-shaped silver nanoparticles.

12. The personal care composition of claim 1, wherein the coral-shaped metal nanoparticles comprise gold nanoparticles.

13. The personal care composition of claim 1, wherein the personal care product is selected from the group consisting of sprays, serums, oils, gels, creams, lotions, emulsions, and semi-solids.

14. A personal care composition, comprising:
a carrier selected from the group consisting of a spray, serum, oil, gel cream, lotion, emulsion, or semi-solid;
coral-shaped gold nanoparticles dispersed throughout the carrier and forming a nanoparticle stabilizing matrix within the carrier, wherein the coral-shaped metal nanoparticles are included at a concentration of at least 0.5 ppm by weight; and
spherical-shaped silver nanoparticles dispersed throughout the carrier and stabilized by the nanoparticle stabilizing matrix, wherein the spherical-shaped metal nanoparticles are included at a concentration of less than 1 ppm by weight, with the proviso that the personal care composition includes a ratio of coral-shaped gold nanoparticles to spherical-shaped silver nanoparticles of greater than 1:1 on a weight basis.

15. The personal care composition of claim 14, wherein the carrier comprises water and at least one hydrophobic component or gelling agent.

16. A method of manufacturing a personal care composition, comprising:
making or proving a carrier comprised of at least one hydrophobic component or gelling agent and optionally water;
dispersing coral-shaped metal nanoparticles throughout the carrier to form a nanoparticle stabilizing matrix within the carrier; and
dispersing spherical-shaped metal nanoparticles throughout the carrier, wherein the spherical metal nanoparticles are suspended and stabilized in the carrier by the nanoparticle stabilizing matrix formed by the coral-shaped metal nanoparticles.

17. The method of claim 16, wherein the coral-shaped metal nanoparticles and the spherical-shaped metal nanoparticles are added to the carrier in amounts so that the personal care composition has a weight ratio of coral-shaped metal nanoparticles to spherical-shaped metal nanoparticles in a range from greater than 1:1 to about 50:1, or from about 1.5:1 to about 25:1, or from about 2:1 to about 15:1, or from about 3:1 to about 10:1.

18. The method of claim 16, wherein the spherical-shaped metal nanoparticles are added to the carrier in an amount so that the personal care composition includes the spherical-shaped metal nanoparticles at a concentration of less than 1 ppm, or in a range from about 2 ppb to about 900 ppb, or from about 10 ppb to about 800 ppb, or from about 20 ppb to about 700 ppb, or from about 30 ppb to about 600 ppb.

19. The method of claim 16, wherein the coral-shaped metal nanoparticles are added to the carrier in an amount so that the personal care composition includes the coral-shaped metal nanoparticles at a concentration of at least 1 ppm, or in a range from about 2 ppm to about 300 ppm, or about 3 ppm to about 200 ppm, or about 4 ppm to about 150 ppm, or about 5 ppm to about 100 ppm.

20. The method of claim 16, wherein the carrier is formulated so that the personal care product is selected from the group consisting of sprays, serums, oils, gels, creams, lotions, emulsions, and semi-solids.
